(19) United States
(12) Patent Application Publication
(10) Pub. No.: US 2018/0161010 A1
(43) Pub. Date: Jun. 14, 2018

(54) APPARATUS AND METHOD FOR PROCESSING ULTRASOUND IMAGE

(71) Applicant: SAMSUNG ELECTRONICS CO., LTD., Suwon-si (KR)

(72) Inventors: Choong-hwan Choi, Seongnam-si (KR); Jong-hyon Yi, Yongin-si (KR); Gun-woo Lee, Seoul (KR)

(73) Assignee: SAMSUNG ELECTRONICS CO., LTD., Suwon-si (KR)

(21) Appl. No.: 15/835,930

(22) Filed: Dec. 8, 2017

(30) Foreign Application Priority Data
Dec. 9, 2016 (KR) 10-2016-0168005

(51) Int. Cl. A61B 8/00; A61B 8/08

(52) U.S. Cl. CPC A61B 8/465; A61B 8/4405; G06F 3/04847; A61B 8/4427; A61B 8/0866

(57) ABSTRACT

Provided are an ultrasound image processing method and an ultrasound image processing apparatus. The ultrasound image processing apparatus includes: an ultrasonic probe configured to acquire ultrasound image data with respect to an object by transmitting ultrasound waves to the object; at least one processor configured to generate at least one ultrasound image based on the ultrasound image data, configured to determine, based on the at least one ultrasound image, whether at least one target region included in an imaging list has been imaged, and generate first imaging status information indicating whether the at least one target region has been imaged; and a display configured to display the first imaging status information.

### CROSS-REFERENCE TO RELATED APPLICATION

This application claims priority from Korean Patent Application No. 10-2016-0168005, filed on Dec. 9, 2016, in the Korean Intellectual Property Office, the disclosure of which is incorporated herein in its entirety by reference.

### BACKGROUND

### 1. Field

Apparatuses and methods consistent with exemplary embodiments relate to ultrasound image processing apparatuses, ultrasound image processing methods, and computer-readable recording media having recorded thereon a program for performing the ultrasound image processing methods.

### 2. Description of the Related Art

Ultrasound image processing apparatuses transmit ultrasound signals generated by transducers of a probe to an object and detect information about signals reflected from the object, thereby obtaining at least one image of an internal part, for example, soft tissue or blood flow, of the object.

The ultrasound image processing apparatuses provide high stability, display images in real time, and are safe because of no radiation exposure, compared to X-ray diagnostic apparatuses. Therefore, the ultrasound image processing apparatuses are widely used together with other types of imaging diagnostic apparatuses.

A precision fetal ultrasound scan in obstetrics and gynecology is performed at six months of pregnancy to check whether a fetus is growing at a rate expected for its gestational age and whether the shape of each organ appears normal and each organ is functioning properly. Unlike other ultrasound examinations performed intensively on a specific body part of a fetus, the precision fetal ultrasound scan is used to check normal growth and development of each body part of a fetus. Thus, during the ultrasound scan, all body parts of the fetus should be scrutinized carefully. Furthermore, in abdominal ultrasound or a gynecological exam performed during medical examination, it is necessary to thoroughly capture images of all predefined body parts for accurate health diagnosis. However, since images need to be captured on a large number of body parts, human error may occur in various ways, such as failing to capture images of some of the body parts or capturing a poor quality image of the body parts.

### SUMMARY

Provided are methods and apparatuses for generating imaging status information based on at least one acquired ultrasound image and an imaging list.

In detail, provided are methods and apparatuses for generating imaging status information indicating whether target regions included in an imaging list have been imaged.

Provided are methods and apparatuses for detecting an ultrasound image corresponding to a target region in an imaging list among acquired at least one ultrasound image and generating imaging status information indicating whether a quality value for the detected ultrasound image is less than a reference value.

Provided are methods and apparatuses for generating imaging status information indicating the progression of imaging being performed on all target regions in an imaging list based on acquired at least one ultrasound image.

Additional aspects will be set forth in part in the description which follows and, in part, will be apparent from the description, or may be learned by practice of the presented embodiments.

According to an aspect of an embodiment, an ultrasound image processing apparatus includes: an ultrasonic probe configured to acquire ultrasound image data with respect to an object by transmitting ultrasound waves to the object; at least one processor configured to generate at least one ultrasound image based on the ultrasound image data, configured to determine, based on the at least one ultrasound image, whether at least one target region included in an imaging list has been imaged, and generate first imaging status information indicating whether the at least one target region has been imaged; and a display configured to display the first imaging status information.

According to an aspect of another embodiment, an ultrasound image processing method includes: acquiring ultrasound image data with respect to an object by transmitting ultrasound waves to the object; generating at least one ultrasound image based on the ultrasound image data; determining, based on the at least one ultrasound image, whether at least one target region included in an imaging list has been imaged, and generate first imaging status information indicating whether the at least one target region has been imaged; and displaying the first imaging status information.

According to an aspect of another embodiment, a computer-readable recording medium has recorded thereon a program for performing an ultrasound image processing method on a computer, the ultrasound image processing method including: acquiring ultrasound image data with respect to an object by transmitting ultrasound waves to the object; generating at least one ultrasound image based on the ultrasound image data; determining, based on the at least one ultrasound image, whether at least one target region included in an imaging list has been imaged, and generate first imaging status information indicating whether the at least one target region has been imaged; and displaying the first imaging status information.

### BRIEF DESCRIPTION OF THE DRAWINGS

These and/or other aspects will become apparent and more readily appreciated from the following description of the embodiments, taken in conjunction with the accompanying drawings in which:

FIG. 1 is a block diagram illustrating an ultrasound image processing apparatus according to an exemplary embodiment;

FIGS. 2A, 2B, and 2C are diagrams respectively illustrating an ultrasound image processing apparatus according to an exemplary embodiment;

FIG. 3 is a block diagram of a configuration of an ultrasound image processing apparatus according to an embodiment;

FIG. 4 is a block diagram of a configuration of an ultrasound image processing apparatus according to another embodiment;

FIG. 5 is a diagram for explaining a process of acquiring first imaging status information and second imaging status information, according to an embodiment;

FIGS. 6A and 6B are exemplary diagrams for explaining a method of displaying first imaging status information on a display, according to embodiments;

FIG. 7 is an exemplary diagram for explaining a method of displaying second imaging status information on a display, according to an embodiment;

FIG. 8 is an exemplary diagram for explaining a method of displaying third imaging status information on a display, according to an embodiment;

FIG. 9 is an exemplary diagram for explaining a method of displaying a first sub-list on a display, according to an embodiment;

FIG. 10 is an exemplary diagram for explaining a method of displaying a second sub-list on a display, according to an embodiment;

FIGS. 11A through 11D are exemplary diagrams for explaining a method of displaying a second sub-list on a display, according to other embodiments;

FIG. 12 is a flowchart of an ultrasound image processing method according to an embodiment;

FIG. 13 illustrates an imaging list according to an embodiment; and

FIG. 14 illustrates an imaging list according to another embodiment.

### DETAILED DESCRIPTION

Certain exemplary embodiments are described in greater detail below with reference to the accompanying drawings.

In the following description, the same drawing reference numerals are used for the same elements even in different drawings. The matters defined in the description, such as detailed construction and elements, are provided to assist in a comprehensive understanding of exemplary embodiments. Thus, it is apparent that exemplary embodiments can be carried out without those specifically defined matters. Also, well-known functions or constructions are not described in detail since they would obscure exemplary embodiments with unnecessary detail.

Terms such as “part” and “portion” used herein denote those that may be embodied by software or hardware. According to exemplary embodiments, a plurality of parts or portions may be embodied by a single unit or element, or a single part or portion may include a plurality of elements. Expressions such as “at least one of,” when preceding a list of elements, modify the entire list of elements and do not modify the individual elements of the list.

In exemplary embodiments, an image may include any medical image acquired by various medical imaging apparatuses such as a magnetic resonance imaging (MRI) apparatus, a computed tomography (CT) apparatus, an ultrasound imaging apparatus, or an X-ray apparatus.

Also, in the present specification, an “object”, which is a thing to be imaged, may include a human, an animal, or a part thereof. For example, an object may include a part of a human, that is, an organ or a tissue, or a phantom.

Throughout the specification, an ultrasound image refers to an image of an object processed based on ultrasound signals transmitted to the object and reflected therefrom.

Throughout the specification, an “imaging list” refers to a list including at least one target region of an object that needs to be imaged for performing a specific test. For example, the imaging list may be a list including target regions that need to be imaged during a precision fetal ultrasound scan and standard views of the target regions.

Throughout the specification, “imaging status information” refers to imaging status information regarding target regions included in an imaging list, which includes pieces of information such as a target region of which imaging is completed, a target region of which imaging has been mistakenly omitted, a quality value for an acquired ultrasound image, progression of imaging being performed on the entire imaging list, etc.

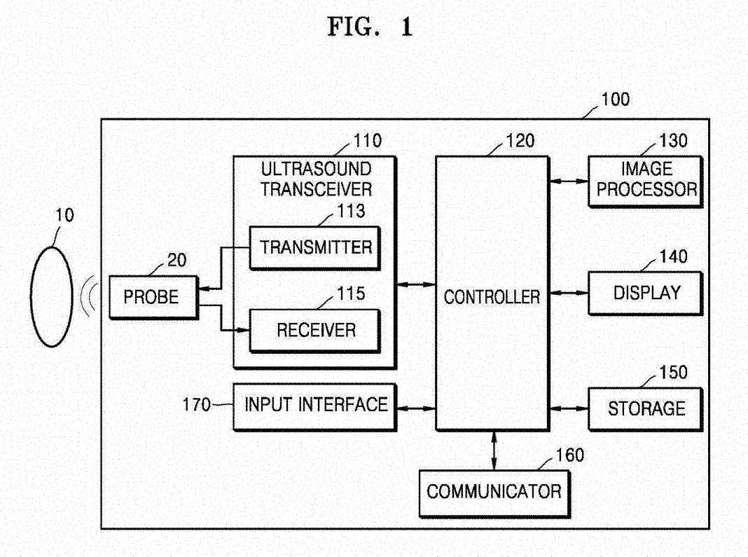

FIG. 1 is a block diagram illustrating a configuration of an ultrasound image processing apparatus 100, i.e., a diagnostic apparatus, according to an exemplary embodiment.

Referring to FIG. 1, the ultrasound image processing apparatus 100 may include a probe 20, an ultrasound transceiver 110, a controller 120, an image processor 130, a display 140, a storage 150, e.g., a memory, a communicator 160, i.e., a communication device or an interface, and an input interface 170.

The ultrasound image processing apparatus 100 may be of a cart-type or a portable-type ultrasound image processing apparatus, that is portable, moveable, mobile, and/or hand-held. Examples of the portable-type ultrasound image processing apparatus 100 may include a smart phone, a laptop computer, a personal digital assistant (PDA), and a tablet personal computer (PC), each of which may include a probe and a software application, but embodiments are not limited thereto.

The probe 20 may include a plurality of transducers. The plurality of transducers may transmit ultrasound signals to an object 10 in response to receiving transmission signals from a transmitter 113. The plurality of transducers may receive ultrasound signals reflected from the object 10 to generate reception signals. In addition, the probe 20 and the ultrasound image processing apparatus 100 may be formed in one body (e.g., disposed in a single housing), or the probe 20 and the ultrasound image processing apparatus 100 may be formed separately (e.g., disposed separately in separate housings) but linked wirelessly or via wires. In addition, the ultrasound image processing apparatus 100 may include one or more probes 20 according to embodiments.

The controller 120 may control the transmitter 113 to generate and transmit the transmission signals to each of the plurality of transducers based on a position and a focal point of the plurality of transducers included in the probe 20.

The controller 120 may control the ultrasound receiver 115 to generate ultrasound data by converting reception signals received from the probe 20 from analog to digital form and summing the reception signals that are converted into digital form, based on a position and a focal point of the plurality of transducers.

The image processor 130 may generate an ultrasound image by using ultrasound data generated from the ultrasound receiver 115.

The display 140 may display a generated ultrasound image and various pieces of information processed by the ultrasound image processing apparatus 100. The ultrasound image processing apparatus 100 may include two or more displays 140 according to an exemplary embodiment. The display 140 may include a touch screen in combination with a touch panel.

The controller 120 may control the operations of the ultrasound image processing apparatus 100 and control flow of signals between the internal elements of the ultrasound image processing apparatus 100. The controller 120 may include a memory for storing a program or data to perform functions of the ultrasound image processing apparatus 100 and a processor and/or a microprocessor (not shown) for processing the program or data. For example, the controller 120 may control the operation of the ultrasound image processing apparatus 100 by receiving a control signal from the input interface 170 or an external apparatus.

The ultrasound image processing apparatus 100 may include the communicator 160 and may be connected to external apparatuses, for example, servers, medical apparatuses, and portable devices such as smart phones, tablet personal computers (PCs), wearable devices, etc., via the communicator 160.

The communicator 160 may include at least one element capable of communicating with the external apparatuses. For example, the communicator 160 may include at least one among a short-range communication module, a wired communication module, and a wireless communication module.

The communicator 160 may receive a control signal and data from an external apparatus and transmit the received control signal to the controller 120 so that the controller 120 may control the ultrasound image processing apparatus 100 in response to the received control signal.

The controller 120 may transmit a control signal to the external apparatus via the communicator 160 so that the external apparatus may be controlled in response to the control signal of the controller 120.

For example, the external apparatus connected to the ultrasound image processing apparatus 100 may process the data of the external apparatus in response to the control signal of the controller 120 received via the communicator 160.

A program for controlling the ultrasound image processing apparatus 100 may be installed in the external apparatus. The program may include command languages to perform at least part of operation of the controller 120 or the entire operation of the controller 120.

The program may be pre-installed in the external apparatus or may be installed by a user of the external apparatus by downloading the program from a server that provides applications. The server that provides applications may include a computer-readable recording medium where the program is stored.

The storage 150 may store various data or programs for driving and controlling the ultrasound image processing apparatus 100, input and/or output ultrasound data, ultrasound images, applications, etc.

The input interface 170 may receive a user's input to control the ultrasound image processing apparatus 100 and may include, for example but not limited to, a keyboard, a button, a keypad, a mouse, a trackball, a jog switch, a knob, a touchpad, a touch screen, a microphone, a motion input means, a biometrics input means, etc. For example, the user's input may include inputs for manipulating buttons, keypads, mice, trackballs, jog switches, or knobs, inputs for touching a touchpad or a touch screen, a voice input, a motion input, and a bio information input, for example, iris recognition or fingerprint recognition, but an exemplary embodiment is not limited thereto.

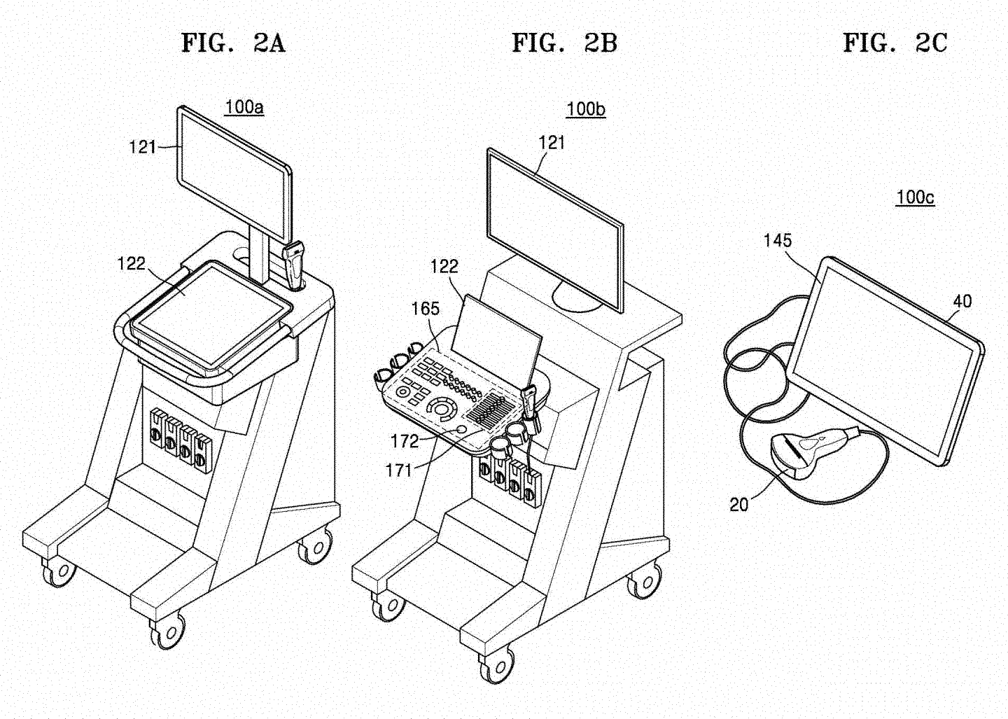

An example of the ultrasound image processing apparatus 100 according to an exemplary embodiment is described below with reference to FIGS. 2A, 2B, and 2C.

FIGS. 2A, 2B, and 2C are diagrams illustrating ultrasound image processing apparatus according to an exemplary embodiment.

Referring to FIGS. 2A and 2B, the ultrasound image processing apparatus 100 may include a main display 121 and a sub-display 122. At least one among the main display 121 and the sub-display 122 may include a touch screen. The main display 121 and the sub-display 122 may display ultrasound images and/or various information processed by the ultrasound image processing apparatus 100. The main display 121 and the sub-display 122 may provide graphical user interfaces (GUI), to receive user's inputs of data or a command to control the ultrasound image processing apparatus 100. For example, the main display 121 may display an ultrasound image and the sub-display 122 may display a control panel to control display of the ultrasound image as a GUI. The sub-display 122 may receive an input of data to control the display of an image through the control panel displayed as a GUI. The ultrasound image processing apparatus 100 may control the display of the ultrasound image on the main display 121 by using the input control data.

Referring to FIG. 2B, the ultrasound image processing apparatus 100 may include a control panel 165. The control panel 165 may include buttons, trackballs, jog switches, or knobs, and may receive data to control the ultrasound image processing apparatus 100 from the user. For example, the control panel 165 may include a time gain compensation (TGC) button 171 and a freeze button 172. The TGC button 171 is to set a TGC value for each depth of an ultrasound image. Also, when an input of the freeze button 172 is detected during scanning an ultrasound image, the ultrasound image processing apparatus 100 may keep displaying a frame image at that time point.

The buttons, trackballs, jog switches, and knobs included in the control panel 165 may be provided as a GUI to the main display 121 or the sub-display 122.

Referring to FIG. 2C, the ultrasound image processing apparatus 100 may include a portable device. An example of the portable ultrasound image processing apparatus 100 may include, for example, smart phones including probes and applications, laptop computers, personal digital assistants (PDAs), or tablet PCs, but an exemplary embodiment is not limited thereto.

The ultrasound image processing apparatus 100 may include the probe 20 and a main body 40. The probe 20 may be connected to one side of the main body 40 by wire or wirelessly. The main body 40 may include a touch screen 145. The touch screen 145 may display an ultrasound image, various pieces of information processed by the ultrasound image processing apparatus 100, and a GUI.

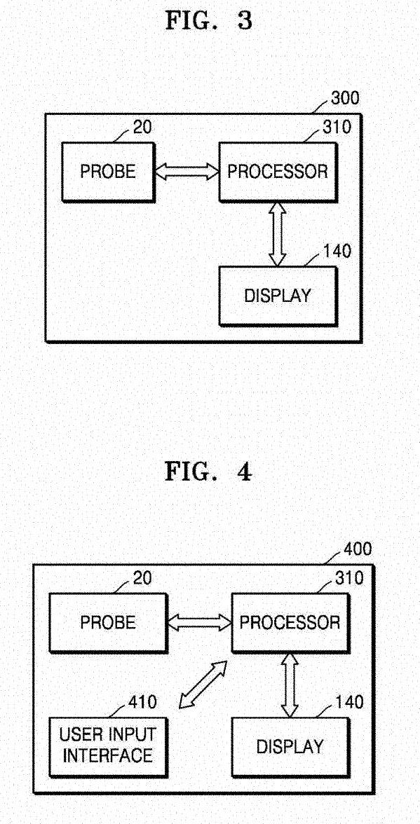

FIG. 3 is a block diagram of a configuration of an ultrasound image processing apparatus 300 according to an embodiment.

Referring to FIG. 3, the ultrasound image processing apparatus 300 according to an exemplary embodiment includes a probe 20, a processor 310, and a display 140.

The processor 310 may correspond to at least one or a combination of the image processor 130 and the controller 120 described with reference to FIG. 1. The processor 310 may include one or more processors (not shown). According to an embodiment, some of the components of the ultrasound image processing apparatus 100 of FIG. 1 may be included in the ultrasound image processing apparatus 300.

The probe 20 transmits ultrasound waves to an object and receives ultrasound echo signals from the object. The probe 20 acquires ultrasound image data based on the received ultrasound echo signals.

According to an embodiment, the probe 20 may transmit ultrasound waves to at least one target region in an imaging list and receive ultrasound echo signals from the at least one target region to acquire ultrasound image data.

The processor 310 controls all or part of operations of the ultrasound image processing apparatus 300 and processes data and signals. According to an embodiment, the processor 310 may include an image processor (not shown) and a controller (not shown). The processor 310 may be implemented as one or more software modules to be executed by program code stored in the storage (150 of FIG. 1).

The processor 310 generates at least one ultrasound image based on ultrasound image data acquired by the probe 20.

The processor 310 detects an ultrasound image corresponding to at least one target region in an imaging list from among the generated at least one ultrasound image.

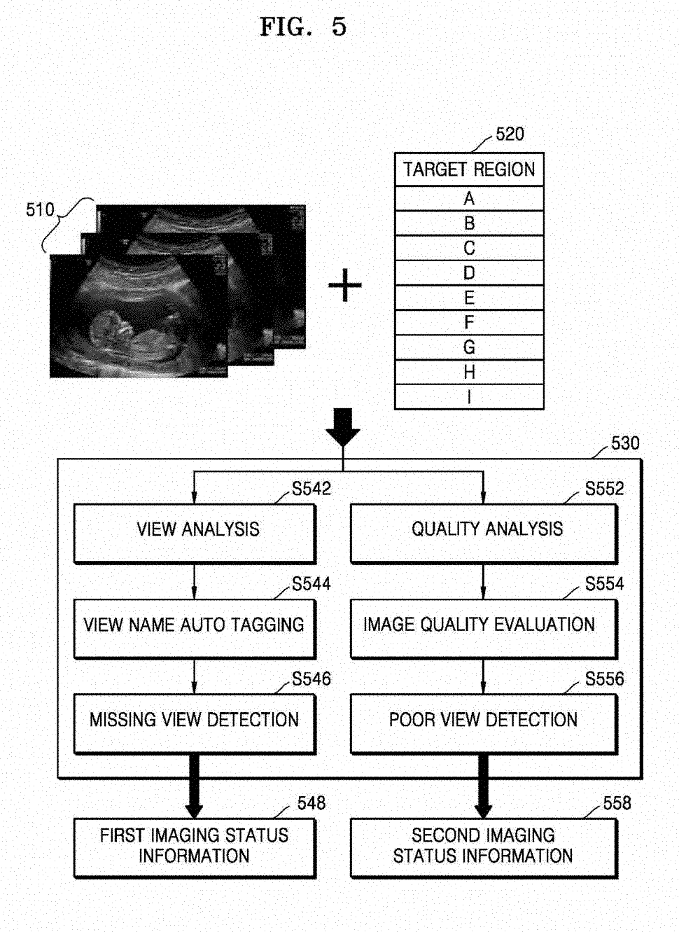

According to an embodiment, the processor 310 may determine whether a target region is shown in a generated ultrasound image, as will be described in more detail below with reference to FIG. 5.

An imaging list means a list including at least one target region of an object that needs to be imaged for performing a specific test.

According to an embodiment, the imaging list may be received from an external server or be determined by the processor 310 based on data acquired from the external server. For example, the processor 310 may receive information about a standard specification or criterion for a specific test from the external server and create an imaging list based on the received information. According to another embodiment, the imaging list may be a list input via a user input interface (e.g., 410 of FIG. 4). According to another embodiment, the imaging list may be a list prestored in the storage 150.

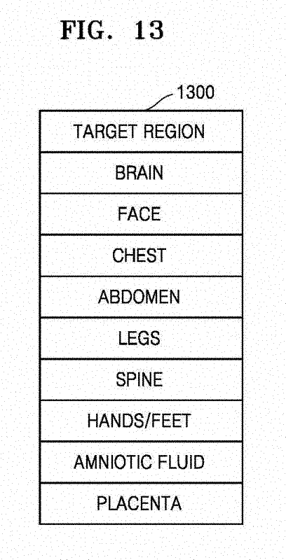

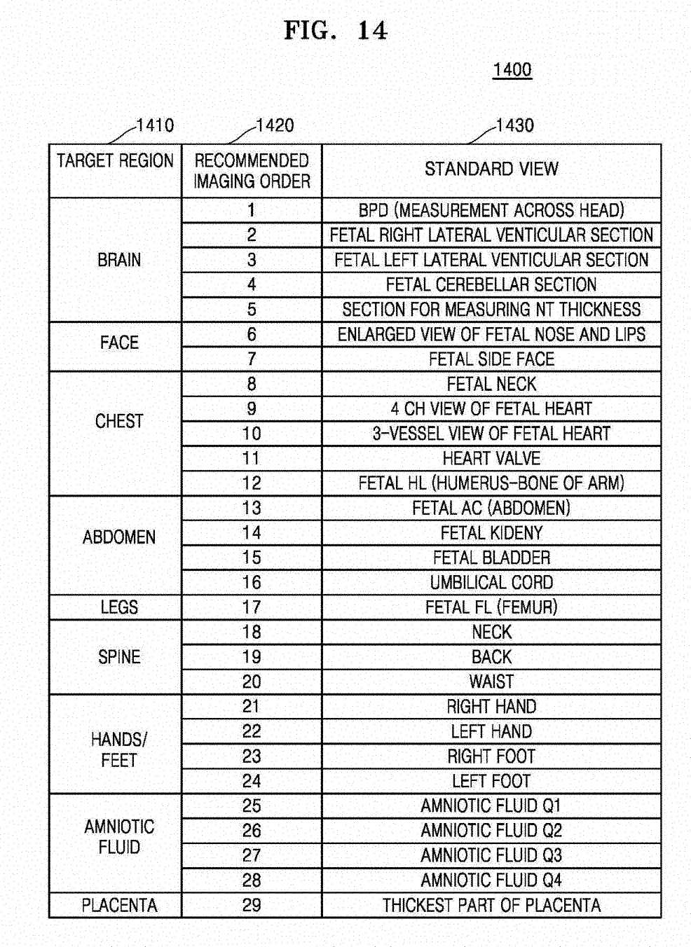

According to an embodiment, the imaging list may include not only a target region of the object but also at least one or a combination of a recommended imaging order and a standard view of the target region. The imaging list will now be described in more detail with reference to FIGS. 13 and 14.

FIG. 13 illustrates an imaging list according to an embodiment.

Referring to FIG. 13, the imaging list may be a list of target regions 1300 of an object that need to undergo ultrasound imaging. For example, when the imaging list is intended for a precision fetal ultrasound scan, the target regions 1300 may include the brain, face, chest, abdomen, legs, spine, hands/feet, amniotic fluid, and placenta.

FIG. 14 illustrates an imaging list 1400 according to another embodiment.

Referring to FIG. 14, the imaging list 1400 may include target regions 1410 of an object, a recommended imaging order 1420, and standard views 1430 of each of the target regions 1410.

The recommended imaging order 1420 may mean an order in which imaging may be efficiently performed on the target regions 1410 or standard views 1430 included in the imaging list 1400. The target regions 1410 or the standard views 1430 may be imaged in the recommended imaging order 1420, such as in the order from the head of the object to the lower limb thereof, in the order from a center of a body of the object to a distal end thereof, or in other orders that enable efficient imaging, so that ultrasound imaging can be efficiently guided.

The standard views 1430 may refer to detailed views of each of the target region of the object that need to be imaged for determining abnormalities of the target regions during a specific test. For example, during a precision fetal ultrasound scan, the standard views 1430 of a target region ‘brain’ may include a fetal biparietal diameter (BPD) (measurement across the head), a fetal right lateral ventricular section, a fetal left lateral ventricular section, a fetal cerebellar section, and a section used to measure a nuchal translucency (NT) thickness.

In the specification, descriptions and configurations related to a ‘target region’ may also be applied to a ‘standard view.’ For example, the processor 310 may detect an ultrasound image corresponding to at least one ‘standard view’ in an imaging list and generate first imaging status information indicating whether the at least one ‘standard view’ has been imaged. The processor 310 may generate second imaging status information indicating whether a quality value for an ultrasound image corresponding to a ‘standard view’ in an imaging list is less than a first reference value and third imaging status information indicating the progression of imaging being performed on all ‘standard views’ in the imaging list. The processor 310 generates first imaging status information indicating whether at least one target region in an imaging list has been imaged.

According to an embodiment, the processor 310 may generate first imaging status information that is used to determine that a target region with respect to which a corresponding ultrasound image is detected from among target regions in an imaging list has been imaged and that a target region with respect to which a corresponding ultrasound image is not detected has not been imaged. By providing the first imaging status information to a user, it is possible to prevent omission of imaging of target regions that have to be imaged, thus ensuring an accurate ultrasound examination.

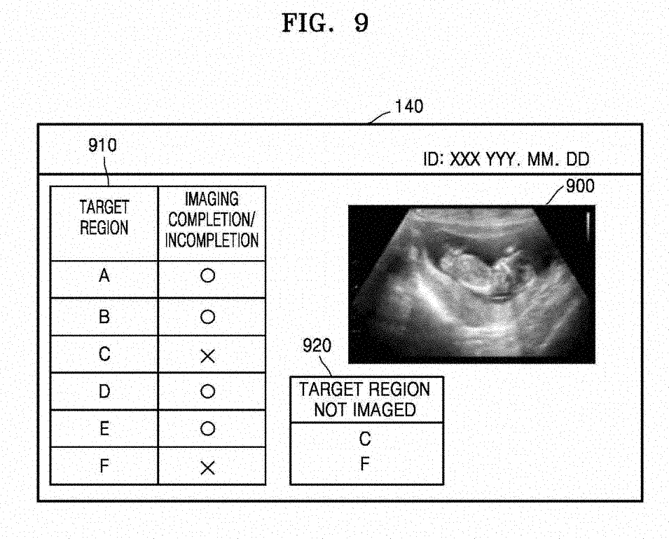

According to an embodiment, the processor 310 may generate, based on the imaging list and the generated first imaging status information, a first sub-list including only target regions that are not imaged among target regions in the imaging list. The first sub-list will be described in more detail below with reference to FIG. 9.

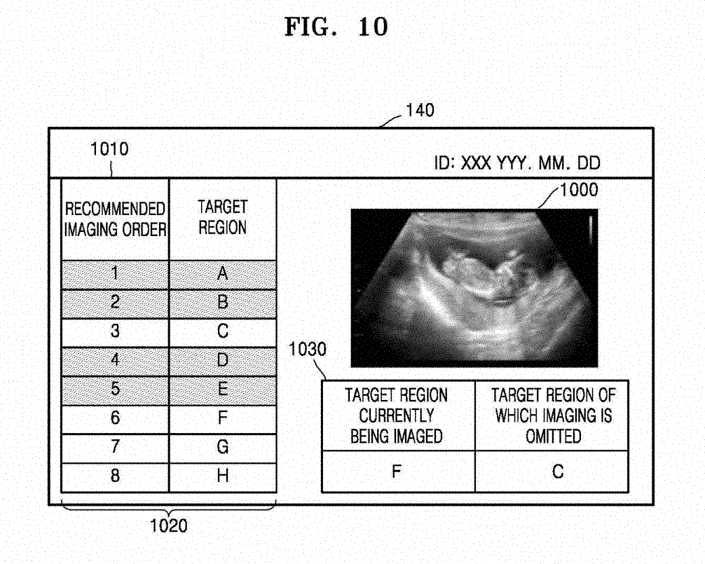

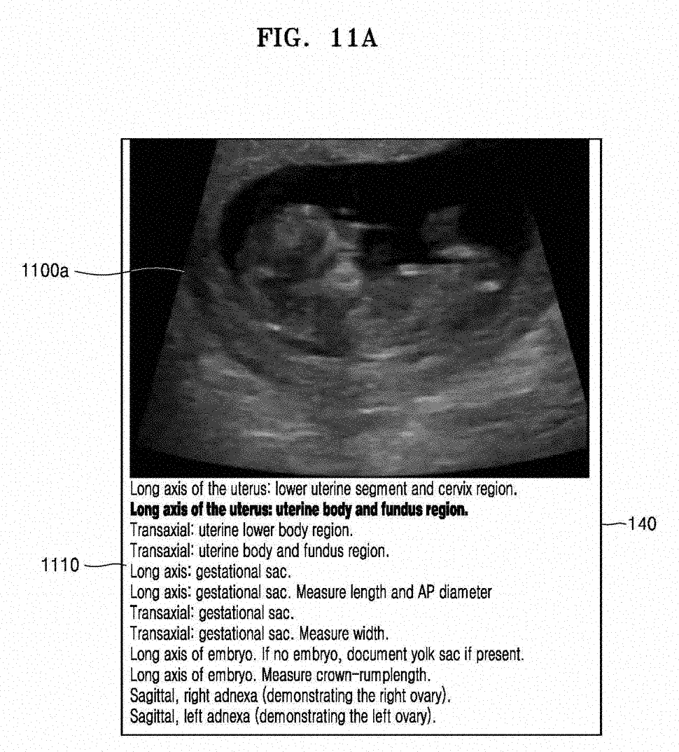

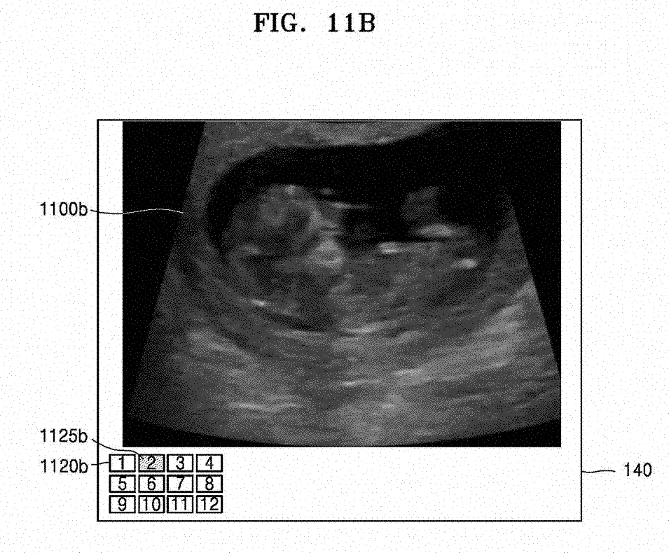

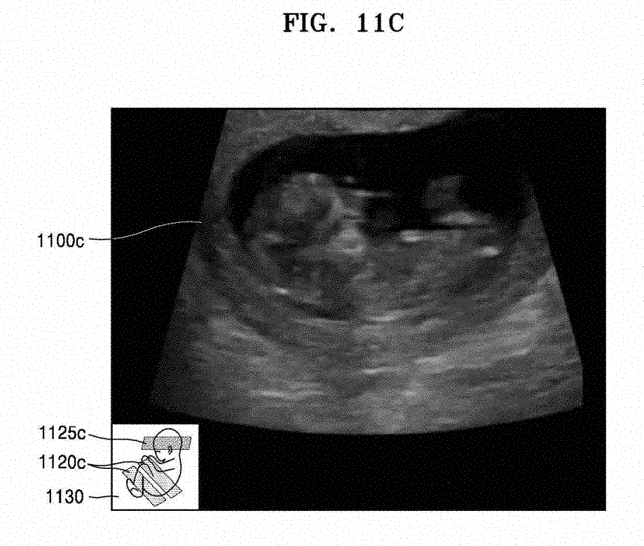

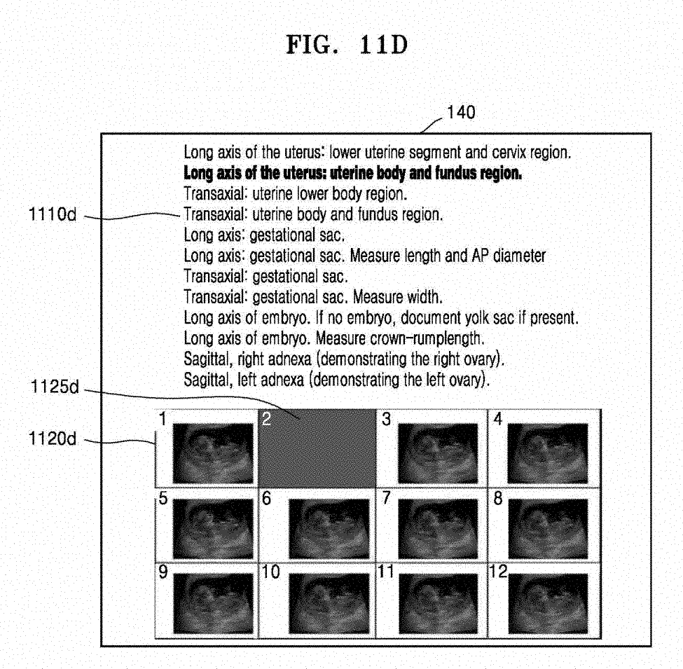

According to an embodiment, when the imaging list includes a recommended imaging order, the processor 310 may generate a second sub-list including at least one of a target region currently being imaged and a target region of which imaging is omitted, based on the recommended imaging order in the imaging list and the first imaging status information. The second sub-list will be described in more detail below with reference to FIGS. 10 and 11A through 11D.

The processor 310 also generates second imaging status information indicating whether a quality value for the detected ultrasound image is less than a predetermined reference value.

According to an embodiment, the processor 310 may calculate a quality value for the detected ultrasound image. A method of calculating a quality value for a detected ultrasound image by determining a quality of the ultrasound image will be described in more detail below with reference to FIG. 5.

According to an embodiment, the processor 310 may set a first reference value as a reference quality measure for an ultrasound image that can be applied to ultrasound diagnosis. The first reference value may be input by the user, be received from an external server, or be calculated by the processor 310 based on a predetermined calculation method.

The processor 310 may generate second imaging status information indicating whether a quality value for an ultrasound image detected for each target region in the imaging list is less than the first reference value. For example, an ultrasound image detected as an image corresponding to a target region in the imaging list may not be used for a required test since the target region is occluded by other organs or may be unsuitable for accurate diagnosis due to much noise contained therein. In this case, by providing the user with information indicating that a quality value for the ultrasound image of the target region is less than a reference value, the processor 310 may control imaging to be performed again.

The processor 310 also generates, based on the imaging list and the first imaging status information, third imaging status information indicating the progression of imaging on all target regions in the imaging list.

According to an embodiment, the processor 310 may calculate, based on the first imaging status information, a percentage (%) of the number of target regions that have been imaged with respect to the total number of target regions in the imaging list. The processor 310 may generate information about the calculated percentage as the third imaging status information. For example, if the total number of target regions in the imaging list is ten (10) and the number of target regions that are determined to have been imaged is four (4), the processor 310 may generate the third imaging status information indicating that 40% of the imaging has been completed. The user may estimate how much of the ultrasound diagnostic process is complete and how much time is left to complete the test based on the third imaging status information.

The display 140 may display an operation state of the ultrasound image processing apparatus 300, an ultrasound image, a user interface screen, etc., based on a control signal from the processor 310.

According to an embodiment, the display 140 may display an ultrasound image generated by the processor 310.

In one embodiment, the display 140 may display an ultrasound image in a first region of a screen and display an imaging list in a second region thereof distinguishable from the first region. In another embodiment, the display 140 may display the imaging list to overlap the entire or a part of the ultrasound image.

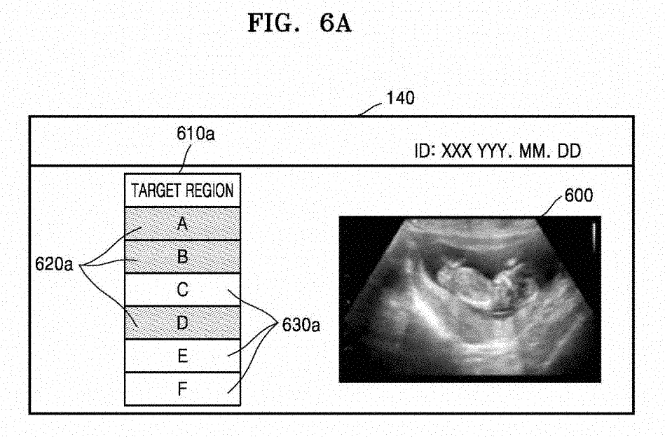

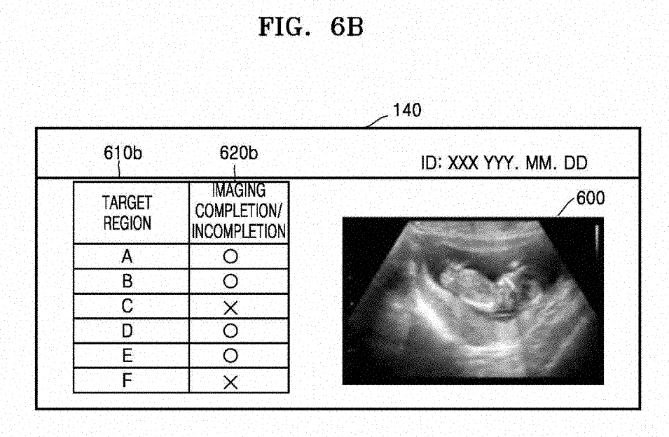

According to an embodiment, the display 140 may display the first imaging status information. A method of displaying the first imaging status information on the display 140 will be described in more detail below with reference to FIGS. 6A and 6B.

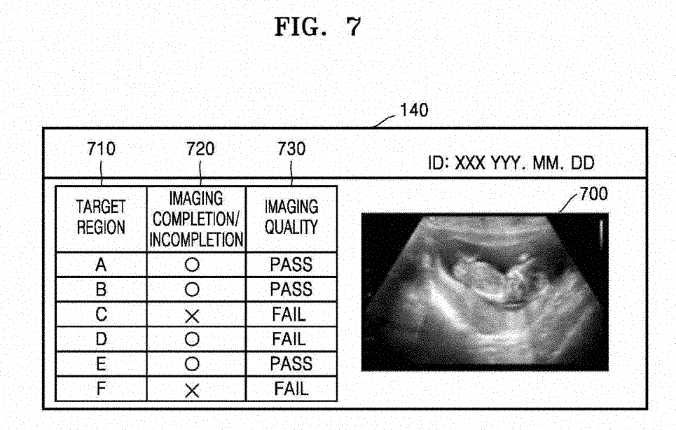

According to an embodiment, the display 140 may display the second imaging status information. A method of displaying the second imaging status information on the display 140 will be described in more detail below with reference to FIG. 7.

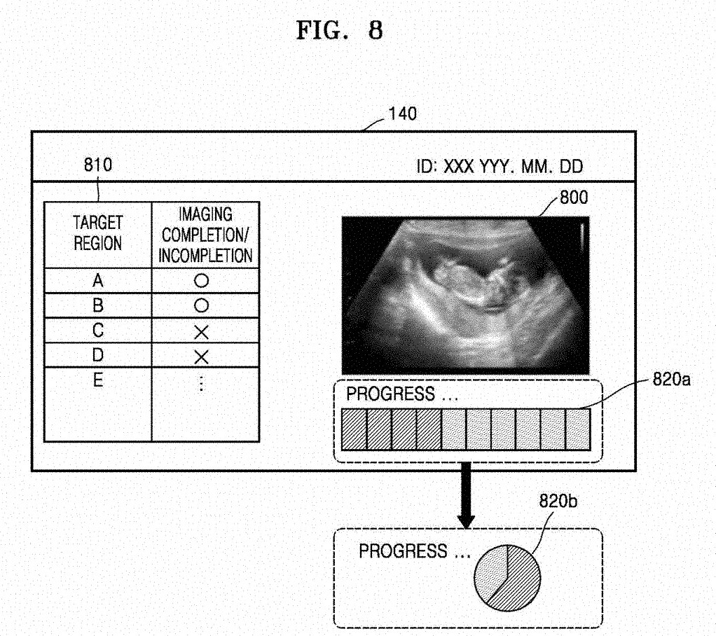

According to an embodiment, the display 140 may display the third imaging status information. A method of displaying the third imaging status information on the display 140 will be described in more detail below with reference to FIG. 8.

According to an embodiment, the display 140 may display a first sub-list. A method of displaying a first sub-list on the display 140 will be described in more detail below with reference to FIG. 9.

According to an embodiment, the display 140 may display a second sub-list. A method of displaying a second sub-list on the display 140 will be described in more detail below with reference to FIG. 10.

FIG. 4 is a block diagram of a configuration of an ultrasound image processing apparatus 400 according to another embodiment.

Referring to FIG. 4, compared with the ultrasound image processing apparatus 300 of FIG. 3, the ultrasound image processing apparatus 400 according to an exemplary embodiment may further include a user input interface 410. The user input interface 410 may correspond to the input interface 170 described with reference to FIG. 1.

The user input interface 410 may receive editing information regarding at least one target region in an imaging list.

According to an embodiment, the user input interface 410 may receive an input for deleting a target region from or adding a new target region to the imaging list.

According to an embodiment, the user input interface 410 may edit the order of arranging target regions in the imaging list. When the imaging list includes a recommended imaging order, the user may edit the recommended imaging order according to a status of imaging. For example, when it is difficult to obtain an ultrasound image of a specific target region due to movement of a fetus during a precision fetal ultrasound scan, the user may edit a recommended imaging order in such a manner as to skip the target region of which imaging is impossible or difficult to perform and capture an image of a target region of which imaging is possible or easier to perform.

In one embodiment, the ultrasound image processing apparatus 400 may further include the communicator (160 of FIG. 1). The communicator 160 may transmit at least one of pieces of the first imaging status information, the second imaging status information, and the third imaging status information generated by the ultrasound image processing apparatus 400 to an external device. The communicator 160 may transmit at least one of first and second sub-lists generated by the ultrasound image processing apparatus 400 to an external device.

FIG. 5 is a diagram for explaining a process of acquiring first imaging status information 548 and second imaging status information 558 according to an embodiment.

According to an embodiment, operations shown in FIG. 5 may be performed by at least one of the ultrasound image processing apparatus 100 shown in FIG. 1), the image processing apparatuses 100a through 100c shown in FIG. 2A through FIG. 2C), the image processing apparatus 300 as shown in FIG. 3, and image processing apparatus 400 shown in FIG. 4. For illustrative purposes, a process, performed by the ultrasound image processing apparatus 300, of acquiring the first imaging status information 548 and the second imaging status information 558 will now be described in detail.

According to an embodiment, the ultrasound image processing apparatus 300 may generate the first imaging status information 548 and the second imaging status information 558 based on ultrasound images 510 and an imaging list 520. Referring to FIG. 5, an algorithm 530 for generating the first imaging status information 548 and the second imaging status information 558 may include operations S542, S544, and S546 and operations S552, S554, and S556. For example, the operations S542, S544, and S546 may be performed parallel with the operations S552, S554, and S556. According to an embodiment, software modules respectively corresponding to the operations included in the algorithm 530 may be implemented by the processor 310 to perform corresponding operations.

The operations S542, S544, and S546 of an algorithm for generating the first imaging status information 548 are described.

In operation S542, the ultrasound image processing apparatus 300 analyzes target regions respectively included in the ultrasound images 510 (View Analysis).

For example, the ultrasound image processing apparatus 300 may extract feature data from the generated ultrasound images 510 and identify anatomical structures based on the feature data. Alternatively, the ultrasound image processing apparatus 300 may identify anatomical structures depicted in the ultrasound images 510 by respectively comparing the ultrasound images 510 with template images of the target regions.

In operation S544, the ultrasound image processing apparatus 300 may automatically tag, based on the identified anatomical structures, the ultrasound images 510 with pieces of information about the target regions included in the ultrasound images 510 (View Name Auto Tagging).

In operation S546, the ultrasound image processing apparatus 300 may detect a target region of which imaging is omitted among target regions in the imaging list 520 based on the pieces of information with which the ultrasound images 510 are automatically tagged (Missing View Detection).

The ultrasound image processing apparatus 300 may detect, based on the pieces of information that are tagged with the ultrasound images 510, an ultrasound image corresponding to a target region in the imaging list 520 from among the ultrasound images 510.

The ultrasound image processing apparatus 300 may generate, based on information about the target region detected as having not been imaged in operation S546, the first imaging status information 548 indicating whether target regions in the imaging list 520 have been imaged.

The operations S552, S554, and S556 of an algorithm for generating the second imaging status information 558 are now described.

In operation S552, The ultrasound image processing apparatus 300 may perform image quality analysis on the ultrasound images 510 (Quality Analysis).

Reference measures such as a signal-to-noise ratio (SNR) and a peak signal-to-noise ratio (PSNR) may be used to perform the image quality analysis.

In operation S554, the ultrasound image processing apparatus 300 may evaluate quality values for the ultrasound images 510 (Image Quality Evaluation).

The quality values for the ultrasound images 510 may be expressed as a quality level or quality score according to a quality measure determined within a predefined value range.

In operation S556, the ultrasound image processing apparatus 300 detects an ultrasound image having a low quality from among the detected ultrasound images 510 (Poor View Detection).

The ultrasound image processing apparatus 300 may acquire a first reference value that is a reference quality measure of the ultrasound images 510 that can be used for ultrasound diagnosis. The first reference value may be input by the user, be received from an external server, or be calculated by the processor 310 based on a predetermined method. The ultrasound image processing apparatus 300 may determine whether the quality values of the ultrasound images 510 are less than the first reference value and detect the ultrasound image 510 having a quality value less than the reference value as being a low quality image.

The ultrasound image processing apparatus 300 may generate, based on detected information about the ultrasound image having the low quality in operation S556, the second imaging status information 558 indicating whether quality values for the ultrasound images 510 detected with respect to target regions in the imaging list 520 are less than the first reference value.

FIGS. 6A and 6B are exemplary diagrams for explaining a method of displaying first imaging status information on the display 140, according to embodiments.

Referring to FIGS. 6A and 6B, the ultrasound image processing apparatus 300 may display an ultrasound image 600 and an imaging list 610a or 610b on the display 140 or a screen of the display 140.

Although FIGS. 6A and 6B show that the ultrasound image 600 and the imaging list 610a or 610b are displayed in regions of the display 140 that are distinguishable from each other, embodiments are not limited thereto. For example, according to an embodiment, the imaging list 610a or 610b may be displayed to overlap an entire or a part region of the acquired ultrasound image 600. The ultrasound image processing apparatus 300 may display the imaging list 610a or 610b in a region of the display 140 corresponding to a user's input. For example, the user may input information about a position at which the imaging list 610a or 610b is to be displayed to the ultrasound image processing apparatus 300 so that the imaging list 610a or 610b may be displayed in a desired screen region. The ultrasound image processing apparatus 300 may receive editing information regarding at least one of a size and a transparency of the imaging list 610a or 610b from the user and display the imaging list 610a or 610b having at least one of a size and a transparency adjusted according to the received editing information.

Referring to FIG. 6A, the ultrasound image processing apparatus 300 may indicate on the imaging list 610 first imaging status information indicating whether at least one target region in the imaging list 610a has been imaged.

According to an embodiment, the ultrasound image processing apparatus 300 may indicate a target region that has been imaged on the imaging list 610a to be distinguishable from a target region that has not been imaged. For example, the ultrasound image processing apparatus 300 may perform shading on the target region that has been imaged on the imaging list 610a. Referring to FIG. 6A, target regions A, B, and D shaded on the imaging list 610a may represent target regions that have been imaged while unshaded target regions C, E, and F may represent target regions that have not been imaged. In another embodiment, the ultrasound image processing apparatus 300 may display the target regions that have been imaged and those that have not been imaged in different text or background colors in such a manner that they are distinguishable from each other.

Referring to FIG. 6B, the ultrasound image processing apparatus 300 may display the first imaging status information indicating whether at least one target region in the imaging list 610b has been imaged or not on a separate imaging completion/incompletion list 620b that is distinguishable from the imaging list 610b.

According to an embodiment, the ultrasound image processing apparatus 300 may generate the imaging completion/incompletion list 620 that is distinguishable from the imaging list 610b and display the first imaging status information on the imaging completion/incompletion list 620b. Referring to FIG. 6B, target regions A, B, D, and E indicated by reference character ‘O’ may represent target regions that have been imaged while target regions C and F indicted by reference character ‘X’ may represent target regions that have not been imaged. In other embodiments, the ultrasound image processing apparatus 300 may indicate imaging completion or incompletion on the imaging completion/incompletion list 620b by using marks other than reference characters O and X. For example, the ultrasound image processing apparatus 300 may distinctively indicate the target regions that have been imaged and those that have not been imaged on a separate list that is distinguishable from the imaging list 610b by using graphical indicators such as checkboxes, geometrical shapes, colors, icons, etc.

According to an embodiment, the ultrasound image processing apparatus 300 may be configured to automatically detect an ultrasound image corresponding to a target region in the imaging list 610a or 610b and generate and display first imaging status information based on a result of the detecting, thereby allowing the user to easily recognize a target region that has not been imaged among target regions in the imaging list 610a or 610b. This configuration may prevent omission of imaging due to human errors that may occur during an ultrasound scan for acquiring a large number of images of target regions or standard views, thereby improving the accuracy of ultrasound scan.

FIG. 7 is an exemplary diagram for explaining a method of displaying second imaging status information on the display 140 or a screen of the display 140, according to an embodiment.

An imaging list 710 shown in FIG. 7 may correspond to the imaging lists 610a and 610b respectively described with reference to FIGS. 6A and 6B, and repetitive descriptions provided above with respect to FIGS. 6A and 6B will be omitted here. For illustrative purposes, FIG. 7 shows that first imaging status information is displayed as an imaging completion/incompletion list 720 that corresponds to the imaging list 620b described with reference to FIG. 6B. However, embodiments are not limited thereto, and the first imaging status information may be displayed in a list corresponding to the imaging list 610a shown in FIG. 6A or in any other various ways as described with reference to FIGS. 6A and 6B.

Referring to FIG. 7, the ultrasound image processing apparatus 300 may display as an imaging quality list 730 second imaging status information indicating whether quality values of ultrasound images corresponding to target regions in the imaging list 710 are less than a predetermined reference value.

For example, in a case where a quality value of an ultrasound image 700 corresponding to a target region in the imaging list 710 is less than a first reference value, the ultrasound image processing apparatus 300 may indicate ‘FAIL’ in the imaging quality list 730 with respect to the corresponding target region. In a case where the quality value thereof is greater than or equal to the first reference value, the ultrasound image processing apparatus 300 may indicate ‘PASS’ in the imaging quality list 730 with respect to the corresponding target region. The ultrasound image processing apparatus 300 may indicate whether a quality value of the ultrasound image 700 is less than the first reference value by using various graphical indicators other than ‘PASS’ and ‘FAIL’, such as geometrical shapes, colors, checkboxes, icons, etc. In an embodiment, the ultrasound image processing apparatus 300 may indicate ‘FAIL’ with respect to a region of which imaging has not been completed. However, embodiments are not limited thereto, and the ultrasound image processing apparatus 300 may not indicate ‘PASS’ or ‘FAIL’ or any quality value with respect to a region of which imaging has not been completed.

According to an embodiment, the ultrasound image processing apparatus 300 may display the second imaging status information via a separate user interface. For example, when a quality value of an acquired ultrasound image corresponding to a target region is determined to be less than the first reference value, the ultrasound image processing apparatus 300 may output a notification window indicating that the user may repeat imaging on the target region.

FIG. 8 is an exemplary diagram for explaining a method of displaying third imaging status information on the display 140 or a screen of the display 140, according to an embodiment.

Referring to FIG. 8, the ultrasound image processing apparatus 300 may display, based on a detected ultrasound image 800, the third imaging status information indicating progression of imaging on all target regions in an imaging list 810 as a progress bar 820a or pie chart 820b. Based on the imaging list 810 and first imaging status information (e.g., an imaging completion/incompletion list), it is determined that target regions A and B among all target regions A through E in the imaging list 810 have been imaged while target regions C, D, and E have not been imaged. When the target region E is currently being imaged, since imaging of the two (2) target regions A and B among a total of five (5) target regions is completed, the ultrasound image processing apparatus 300 may display the third imaging status information as the progress bar 820a or pie chart 820b indicating that about 40% of the ultrasound imaging has been completed.

According to an embodiment, the ultrasound image processing apparatus 300 may display the third imaging status information other than the press bar 820a or the pie chart 820b, for example, by using numbers, geometrical shapes, or any other various graphs.

According to an embodiment, the ultrasound image processing apparatus 300 may determine, based on a user's input received via the user input interface (e.g., 410 of FIG. 4), a position on the display 140 at which the third imaging status information is to be displayed. The ultrasound image processing apparatus 300 may receive editing information regarding at least one of a size and a transparency of the third imaging status information from the user input interface 410 and display the third imaging status information in such a manner as to correspond to the received editing information (e.g., display the third status information to have the size and/or transparency corresponding to the editing information).

FIG. 9 is an exemplary diagram for explaining a method of displaying a first sub-list 920 on the display 140 or a screen of the display 140, according to an embodiment.

According to an embodiment, the ultrasound image processing apparatus 300 may generate, based on an imaging list 910 and first imaging status information (e.g., an imaging completion/incompletion list), the first sub-list 920 including only target regions that have not been imaged among target regions in the imaging list 910. Referring to FIG. 9, the ultrasound image processing apparatus 300 may display the first sub-list 920 including only target regions C and F that have not been imaged among target regions A through F in the imaging list 910. Although FIG. 9 shows that the first sub-list 920 is displayed in a region distinguishable from an ultrasound image 900 and the imaging list 910, according to an embodiment, the first sub-list 920 may be displayed to overlap the ultrasound image 900 or the imaging list 910 in its entirety or partially or be displayed in a notification window (e.g., a popup window).

According to an embodiment, the ultrasound image processing apparatus 300 may determine, based on a user's input received via the user input interface 410, a position on the display 140 where the first sub-list 920 is to be displayed. The ultrasound image processing apparatus 300 may also receive from the user input interface 410 editing information regarding at least one of a size and a transparency of the first sub-list 920 to be displayed on the display 140 and display the first sub-list 920 in such a manner as to correspond to the received editing information (e.g., display the first sub-list 920 to have the size and/or transparency corresponding to the editing information).

In addition, according to an embodiment, the ultrasound image processing apparatus 300 may transmit the generated first sub-list 920 to an external device including a display.

FIG. 10 is an exemplary diagram for explaining a method of displaying a second sub-list 1030 on the display 140 or a screen of the display 140, according to an embodiment.

Referring to FIG. 10, according to an embodiment, the ultrasound image processing apparatus 300 may perform ultrasound imaging based on a recommended imaging order list 1010 included in an imaging list 1020. The ultrasound image processing apparatus 300 may obtain ultrasound images of target regions in the same order as indicated in the recommended imaging order list 1010 and generate first imaging status information based on the obtained ultrasound images. The ultrasound image processing apparatus 300 may indicate the first imaging status information on the imaging list 1020. Referring to FIG. 10, the ultrasound image processing apparatus 300 may shade target regions that have been imaged on the imaging list 1020 to be distinguishable from target regions that have not been imaged. However, the ultrasound image processing apparatus 300 may indicate the first imaging status information in other various ways as described with reference to FIGS. 6A and 6B, and a detailed description thereof will not be repeated herein.

The ultrasound image processing apparatus 300 may determine, based on the first imaging status information, a target region listed last in the recommended imaging order list 1010 among target regions that have been imaged. The ultrasound image processing apparatus 300 may determine a target region currently being imaged and a target region of which imaging is mistakenly omitted based on the target region determined as being listed last. The ultrasound image processing apparatus 300 may generate the second sub-list 1030 including at least one of the target regions currently being imaged and of which imaging is mistakenly omitted.

For example, referring to FIG. 10, target region E is listed last in the recommended imaging order list 1010 among target regions that have been imaged. Thus, the ultrasound image processing apparatus 300 may determine target region F listed next to the target region E in the recommended imaging order list 1010 as being a target region currently being imaged. Furthermore, the ultrasound image processing apparatus 300 may determine target region C that is listed before the target region E in the recommended imaging order list 1010 but has not been imaged as being a target region of which imaging is mistakenly omitted.

Although FIG. 10 shows that the second sub-list 1030 is displayed in a region that is distinguishable from an ultrasound image 1000 and the imaging list 1020, according to an embodiment, the second sub-list 1030 may be displayed to overlap the ultrasound image 1000 or the imaging list 1020 in its entirety or partially or be displayed in a notification window (e.g., a popup window).

According to an embodiment, the ultrasound image processing apparatus 300 may determine, based on a user's input received via the user input interface 410, a position on the display 140 where the second sub-list 1030 is to be displayed. The ultrasound image processing apparatus 300 may also receive from the user input interface 410 editing information regarding at least one of a size and a transparency of the second sub-list 1030 to be displayed on the display 140 and display the second sub-list 1030 in such a manner as to correspond to the received editing information (e.g., display the second sub-list 1030 to have the size and/or transparency corresponding to the editing information).

In addition, according to an embodiment, the ultrasound image processing apparatus 300 may transmit the generated second sub-list 1030 to an external device, e.g., an external device including a display.

FIGS. 11A through 11D are exemplary diagrams for explaining a method of displaying a second sub-list on the display 140 or a screen of the display 140, according to other embodiments.

Referring to FIG. 11A, the ultrasound image processing apparatus 300 may display a second sub-list on the display 140 as a list 1110. In detail, the ultrasound image processing apparatus 300 may display as the list 1110 the second sub-list including at least one of a target region currently being imaged and a target region of which imaging is omitted. In an embodiment, the ultrasound image processing apparatus 300 may display the list 1110 in a first area of the screen and display an ultrasound image 1100a in a second area of the screen. However, embodiments are not limited thereto, and the list 1110 may be displayed to overlap entirely or partially with the ultrasound image 1100a.

Referring to FIG. 11B, the ultrasound image processing apparatus 300 may display a second sub-list on the display 140 as thumbnail images 1120b. The ultrasound image processing apparatus 300 may generate the thumbnail images 1120b representing ultrasound images corresponding to target regions in an imaging list and display the second sub-list in such a manner that a region 1125b corresponding to a target region of which imaging is omitted is indicated in a color or with shading that is distinguishable from that of the other regions on the thumbnail images 1120b. In an embodiment, the ultrasound image processing apparatus 300 may display the list 1120b in a first area of the screen and display an ultrasound image 1100b in a second area of the screen. However, embodiments are not limited thereto, and the list 1120b may be displayed to overlap entirely or partially with the ultrasound image 1100b.

Referring to FIG. 11C, the ultrasound image processing apparatus 300 may display on a model image 1130 of an object a second sub-list in which regions corresponding to target regions currently being imaged and of which imaging is omitted are respectively indicated by different indicators 1120c and 1125c.

For example, it is assumed that the object is a fetus, a target region currently being imaged is the brain, and target regions of which imaging is omitted are the ‘legs’ and ‘abdomen.’ The ultrasound image processing apparatus 300 may display a second sub-list in which regions corresponding to the ‘brain’ is indicated by an indicator 1125c and regions corresponding to ‘legs’ and ‘abdomen’ are indicated in an indicator 1120c on a model image 1130 of the fetus. The indicator 1125c and the indicator 1120c may be distinguishable from each other by using various forms of graphical indicators such as checkboxes, geometrical shapes, colors, shadings, icons, etc. In an embodiment, the ultrasound image processing apparatus 300 may display the model image 1130 to overlap with an ultrasound image 1100c. However, embodiments are not limited thereto, and the model image 1130 may be displayed on a region of a screen separate from the ultrasound image 1100c.

Referring to FIG. 11D, the ultrasound image processing apparatus 300 may display a second sub-list on the display 140 as a list 1110d and as thumbnail images 1120d. A target region of which imaging is omitted or of which imaging has an image quality lower than a threshold may be represented by an indicator 1125d. Descriptions of methods displaying the second sub-list as the list 1110d and as the thumbnail images 1120d are already provided above with respect to FIGS. 11A and 11B and thus will not be repeated herein.

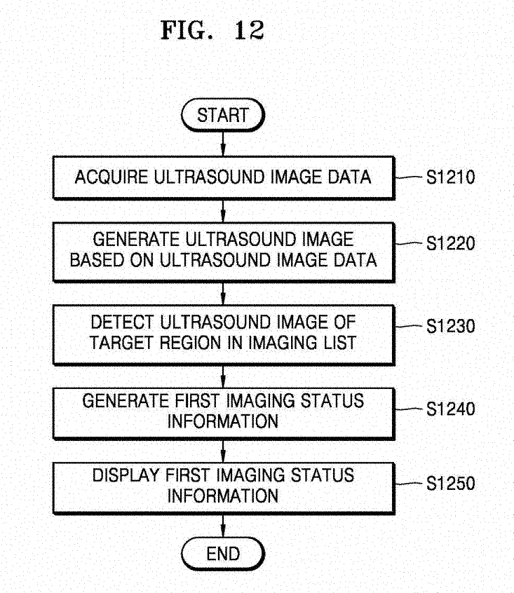

FIG. 12 is a flowchart of an ultrasound image processing method according to an embodiment.

The ultrasound image processing method illustrated in FIG. 12 may be performed by the ultrasound image processing apparatus 100 or 300 or 400, and operations of the method may be the same as those performed by the ultrasound image processing apparatus 100 or 300 or 400 described with reference to FIGS. 1, 3 and 4. Thus, descriptions that are already provided above with respect to FIGS. 1, 3 and 4 will be omitted below. For illustrative purposes, a process, performed by the ultrasound image processing apparatus 300 will now be described in detail.

The ultrasound image processing apparatus 300 transmits ultrasound waves to an object and acquires ultrasound image data with respect to the object (S1210).

The ultrasound image processing apparatus 300 generates at least one ultrasound image based on the ultrasound image data (S1220).

The ultrasound image processing apparatus 300 detects an ultrasound image corresponding to at least one target region in an imaging list from among the generated at least one ultrasound image (S1230).

The ultrasound image processing apparatus 300 generates, based on the ultrasound image detected as being an image corresponding to the at least one target region in the imaging list, first imaging status information indicating whether the at least one target region has been imaged (S1240).

The ultrasound image processing apparatus 300 displays the generated first imaging status information (S1250).

The above-described embodiments of the disclosure may be embodied in form of a computer-readable recording medium for storing computer executable command languages and data. The command languages may be stored in form of program codes and, when executed by a processor, may perform a certain operation by executing a certain program module. Also, when executed by a processor, the command languages may perform certain operations of embodiments.

At least one of the components, elements, modules or units represented by a block as illustrated in the drawings may be embodied as various numbers of hardware, software and/or firmware structures that execute respective functions described above, according to an embodiment. For example, at least one of these components, elements or units may use a direct circuit structure, such as a memory, a processor, a logic circuit, a look-up table, etc. that may execute the respective functions through controls of one or more microprocessors or other control apparatuses. Also, at least one of these components, elements or units may be specifically embodied by a module, a program, or a part of code, which contains one or more executable instructions for performing specified logic functions, and executed by one or more microprocessors or other control apparatuses. Also, at least one of these components, elements or units may further include or be implemented by a processor such as a central processing unit (CPU) that performs the respective functions, a microprocessor, or the like. Two or more of these components, elements or units may be combined into one single component, element or unit which performs all operations or functions of the combined two or more components, elements of units. Also, at least part of functions of at least one of these components, elements or units may be performed by another of these components, element or units. Further, although a bus is not illustrated in the above block diagrams, communication between the components, elements or units may be performed through the bus. Functional aspects of the embodiments may be implemented in algorithms that execute on one or more processors. Furthermore, the components, elements or units represented by a block or processing steps may employ any number of related art techniques for electronics configuration, signal processing and/or control, data processing and the like.

While embodiments of the disclosure have been particularly shown and described with reference to the accompanying drawings, it will be understood by those of ordinary skill in the art that various changes in form and details may be made therein without departing from the spirit and scope of the invention as defined by the appended claims. The disclosed embodiments should be considered in descriptive sense only and not for purposes of limitation.

What is claimed is:

1. An ultrasound image processing apparatus comprising:
an ultrasonic probe configured to acquire ultrasound image data with respect to an object by transmitting ultrasound waves to the object;
at least one processor configured to generate at least one ultrasound image based on the ultrasound image data, configured to determine, based on the at least one ultrasound image, whether at least one target region included in an imaging list has been imaged, and generate first imaging status information indicating whether the at least one target region has been imaged; and
a display configured to display the first imaging status information.

2. The ultrasound image processing apparatus of claim 1, wherein the at least one processor is further configured to generate second imaging status information indicating whether a quality value of an ultrasound image corresponding to the at least one target region is less than a first reference value, and
wherein the display is further configured to display the second imaging status information.

3. The ultrasound image processing apparatus of claim 1, further comprising a user input interface configured to receive editing information regarding the at least one target region in the imaging list.

4. The ultrasound image processing apparatus of claim 1, wherein the at least one processor is further configured to generate, based on the at least one ultrasound image, third imaging status information indicating progression of imaging being performed on target regions in the imaging list, and
wherein the display is further configured to display the third imaging status information.

5. The ultrasound image processing apparatus of claim 1, wherein the imaging list comprises at least one standard view of the at least one target region.

6. The ultrasound image processing apparatus of claim 1, wherein the imaging list comprises a recommended imaging order in which the at least one target region is to be imaged.

7. The ultrasound image processing apparatus of claim 1, wherein the at least one processor is further configured to generate, based on the first imaging status information and the imaging list, a first sub list including a target region that has not been imaged, and
wherein the display is further configured to display the first sub list.

8. The ultrasound image processing apparatus of claim 6, wherein the at least one processor is further configured to generate, based on the recommended imaging order in the imaging list and the first imaging status information, a second sub list including at least one of a target region currently being imaged and a target region of which imaging is omitted, and
wherein the display is further configured to display the second sub list.

9. An ultrasound image processing method comprising:
acquiring ultrasound image data with respect to an object by transmitting ultrasound waves to the object;
generating at least one ultrasound image based on the ultrasound image data;
determining, based on the at least one ultrasound image, whether at least one target region included in an imaging list has been imaged;
generating first imaging status information indicating whether the at least one target region has been imaged; and
displaying the first imaging status information.

10. The ultrasound image processing method of claim 9, further comprising:
generating second imaging status information indicating whether a quality value of an ultrasound image corresponding to the at least one target region is less than a first reference value; and
displaying the second imaging status information.

11. The ultrasound image processing method of claim 9, further comprising receiving editing information regarding the at least one target region in the imaging list.

12. The ultrasound image processing method of claim 9, further comprising:
generating, based on the at least one ultrasound image, third imaging status information indicating progression of imaging being performed on target regions in the imaging list; and
displaying the third imaging status information.

13. The ultrasound image processing method of claim 9, wherein the imaging list comprises at least one standard view of the at least one target region.

14. The ultrasound image processing method of claim 9, wherein the imaging list comprises a recommended imaging order in which the at least one target region is to be imaged.

15. The ultrasound image processing method of claim 9, further comprising:
generating, based on the first imaging status information and the imaging list, a first sub list including a target region that has not been imaged; and
displaying the first sub list.

16. The ultrasound image processing method of claim 14, further comprising:
generating, based on the recommended imaging order in the imaging list and the first imaging status information, a second sub list including at least one of a target region currently being imaged and a target region of which imaging is omitted; and
displaying the second sub list.

17. A computer-readable recording medium, having recorded thereon a program for performing an ultrasound image processing method on a computer, the ultrasound image processing method comprising:
acquiring ultrasound image data with respect to an object by transmitting ultrasound waves to the object;
generating at least one ultrasound image based on the ultrasound image data;
determining, based on the at least one ultrasound image, whether at least one target region included in an imaging list has been imaged;
generating first imaging status information indicating whether the at least one target region has been imaged; and
displaying the first imaging status information.
